(19) United States
(12) Patent Application Publication
(10) Pub. No.: US 2010/0272797 A1
(43) Pub. Date: Oct. 28, 2010

(54) Pharmaceutical Composition for Treating Hepatitis C Virus Infection Comprising HMG-COA Reductase Inhibitor and Bile Acid

(75) Inventors: Sung Wuk Kim, Seongnam-si (KR); Sung Soo Jun, Seongnam-si (KR); Young Gwan Jo, Yuseong-gu (KR); Ja Seong Koo, Yuseong-gu (KR); Jun Young Lee, Yuseong-gu (KR)

Correspondence Address: SWANSON & BRATSCHUN, L.L.C., 8210 SOUTHPARK TERRACE, LITTLETON, CO, 80120, US

(73) Assignee: HANALL PHARMACEUTICAL COMPANY, LTD., Daedeok-gu, Daejeon (KR)

(21) Appl. No.: 12/682,570

(22) PCT Filed: Oct. 10, 2008
(86) PCT No.: PCT/KR08/05965

§ 371 Date: Apr. 9, 2010

(30) Foreign Application Priority Data
Oct. 10, 2007 (KR) 10-2007-0101922

(51) Int. Cl. A61K 31/575; A61K 38/21; A61K 9/00; A61K 9/20; A61K 9/54; A61K 9/28; A61P 31/14

(52) U.S. Cl. 424/458; 514/171; 424/85.4; 424/484; 424/464; 424/474

(57) ABSTRACT

Disclosed herein is a pharmaceutical composition for treating hepatitis C virus infection comprising an HMG-CoA reductase inhibitor and bile acid. More specifically, disclosed are a pharmaceutical composition for treating hepatitis C vims infection comprising fluvastatin or a pharmaceutically acceptable salt thereof along with ursodeoxycholic acid and a preparation method thereof.

Pharmaceutical composition for treating hepatitis C virus infection comprising HMG-CoA reductase inhibitor and bile acid.

### TECHNICAL FIELD

The present invention relates to a pharmaceutical composition for treating. hepatitis C virus infection, comprising an HMG-CoA reductase inhibitor and bile acid.

### BACKGROUND ART

Based on the statistics obtained in the year 2004, it is estimated that about 3% of the world population is infected with hepatitis C virus. Particularly, the prevalence of hepatitis C virus infection in seven countries (USA, Japan, France, Germany, Italy, Spain and England) in 2006, for which accurate statistics are available, is 1.4% corresponding to more than ten million persons among seven hundred million persons. Hepatitis C virus causing this disease is an enveloped, single-stranded RNA virus belonging to the genus Hepacivirus in the family Flaviviridae and infects people mainly through body fluids such as blood.

15-45% of patients infected with this virus recover after suffering from acute hepatitis, but the remaining 55-85% of patients progress to chronic hepatitis, and about 5-20% thereof progress to liver cirrhosis after 20-25 years. Liver cancer develops in 1-5% of those with chronic infection each year.

Prior methods for treating hepatitis C virus infection typically include methods of administering interferons alone and methods of administering interferons in combination with antiviral drugs. Interferons are cytokines produced primarily in the human body and have antiviral action, antiproliferative action and immunomodulating action. Among interferons, interferon-alpha is mainly used to treat hepatitis C and hepatitis B, and as interferon-alpha products, once-daily formulations and once-weekly formulations are used (Roche Pegasys, Schering Peg-Intron, Intron A, etc.).

Interferon therapies may be fundamental therapies capable of completely removing virus, but when interferon is used alone, the treatment rate does not exceed an average of 30%, although it varies depending on a clinical group. However, it has been proved through all clinical experiments that, when interferon and an antiviral drug are used in combination, the treatment rate is higher than that of the interferon single therapy.

A typical one of antiviral drugs (small-molecule drugs) is ribavirin. Ribavirin shows the effects of inhibiting the proliferation of hepatitis C virus and stimulating T cells to increase innate immunity. However, it cannot remove hepatitis virus and when the administration of the drug is discontinued, the blood ALT (alanine aminotransferase) levels are elevated again. Because some hepatitis viruses exist in the body even after the administration of the drug has been completed according to clinical treatment standards, the risk of recurrence of hepatitis virus infection still exists.

Thus, Ribavirin is one of the drug which must be administered for a long-term period, but shows very serious side-effects due to long-term administration. That is a drug which is distributed in hematocytes in large amounts, leading to a very serious side-effect of causing anemia.

In addition, the treatment rate of the combination therapy of interferon and ribavirin is still about 55%. The number of hepatitis C patients is large, and if they are untreated, hepatitis C progresses to liver cirrhosis and liver cancer. For these reasons, a drug which significantly increases the treatment rate of the combination therapy must be developed and, furthermore, the newly developed drug is a safe drug which can be administered for a long-term period.

Fluvastatin which is widely used as a hyperlipidemia therapeutic agent is an HMG-CoA reductase inhibitor and has a chemical name of [R,S-(E)]-(±)-7-[3-(4-fluorophenyl)-1-(1-methylethyl)-1H-indol-2-yl]-3,5-dihydroxy-6-heptanoate, and sodium salts thereof are mainly used. The HMG-CoA reductase inhibitor inhibits fatty acid from being converted to mevalonate by HMG-CoA reductase, thus preventing the synthesis of cholesterol. Accordingly, it reduces blood cholesterol levels, thus treating hypercholesterolemia. Therefore, it is used to prevent and treat cardiovascular diseases caused by the abnormal blood flood of arteries. It is currently well known that fluvastatin is most frequently used among drugs for treating hyperlipidemia.

HMG-CoA reductase inhibitors suppress the initial stage of cholesterol synthesis to reduce the production of the final product cholesterol and substances derived therefrom. Also, in the intermediate stage, they inhibits the synthesis of geranyl phosphate which is a substance allowing hepatitis C virus to proliferate. Thus, HMG-CoA reductase inhibitors can inhibit the proliferation of hepatitis C virus. As such HMG-CoA reductase inhibitors, fluvastatin, lovastatin, atorvastatin, simvastatin, livastatin, pitavastatin, rosuvastatin, and salts thereof are widely used, and among them, fluvastatin shows the strongest efficacy (See Different anti-HCV profiles of statins and their potential for combination therapy with interferon. Ikeda M, Abe K, Yamada M, Dansako H, Naka K, Kato N., Hepatology. 2006 July; 44(1):117-25).

The fluvastatin has an efficacy superior to that of ribavirin that is the prior hepatitis antiviral drug. In addition, it has no serious side effect, and thus can be used for a long-term period. These facts had been reported by a Japanese research group and through Digestive Disease Week (DDW) held on May, 2007 in Washington D.C., USA ( . . . , DDW: Could Statins Be a New Option for Hepatitis C Patients, DDW, 2007, May).

However, about 1.1% of hepatitis patients administered with fluvastatin showed a continuous increase in transaminase levels in proportion to the dose of fluvastatin, and the transaminase levels were elevated to at least 3 times the upper limit of normal. At least 90% of such patients showed an increase in blood transaminase levels within 12 weeks after the administration of fluvastatin. This is because the synthesis of bile acid in the liver is impaired due to virus, so that a metabolic process of conjugating lipid residue and discharging the lipid to fine bile ducts is impaired for a long period of time. When the impairment is not quickly recovered, recovery from hepatitis is delayed.

Bile acids have been used to treat liver function abnormality, chronic hepatitis, liver cirrhosis, defective biliary secretion and the like. Known examples of such bile acids include ursodeoxycholic acid, chenodeoxycholic acid, deoxycholic acid, cholic acid and the like. The fact that the Japanese Health and Welfare Ministry recently approved the application of ursodeoxycholic acid against viral hepatitis supports that bile acids can be used for the treatment of viral hepatitis.

Ursodeoxycholic acid is a kind of bile acid that is found mainly in bear bile, and it is also found in human bile in an amount of about 5%. With respect to the medicinal effects of bear's gall which has been known for a long time as the best medicine against liver diseases, a Swedish research team demonstrated through molecular structural methods for the first time in the world in the 1930s that the main medicinal component of bear's gall is ursodeoxycholic acid. In the year 1961, Japanese scientists artificially synthesized ursodeoxycholic acid in the first time, and the medicinal effects of bear's gall that has been started to demonstrate by the artificial synthetic substances. In 1989, the formal application of ursodeoxycholic acid against primary biliary cirrhosis was approved in France, Germany and England. In the year 1990, the application of ursodeoxycholic acid was approved by the US FDA. In the year 2007, Mitsubishi Pharmaceutical Corporation was approved by the Japanese Health and Welfare Ministry with respect to the application of ursodeoxycholic acid against viral hepatitis. In Korea, the application of ursodeoxycholic acid against cholelithiasis, chronic hepatitis, defective biliary secretion and the like is currently being approved.

It has been reported in the literature that ursodeoxycholic acid has various effects, such as preventing hepatitis B, hepatitis C, liver cirrhosis and liver cancer from growing worse, inhibiting hyperlipidemia and suppressing immunity in organ transplantation. Ursodeoxycholic acid lowers biochemical parameters such as ALT, AST and GGT in chronic hepatitis patients (See C. Sama et al., Clin. Drug. Invest 13(4), 192-198 (1997)) and prevents acute hepatitis B viral infection from becoming chronic (See J. Galsky et al., J. CLIN. Gastroenterol. 28(3). 249-253 (1999)). Also, ursodeoxycholic acid is a therapeutic agent effective in ameliorating chronic inactive hepatitis caused by chronic hepatitis C virus infection which recurred after liver transplantation (See Y. Kita et al., Transpl. pro. 28, 1701-1703 (1996)), and when ursodeoxycholic acid in combination with interferon is administered to chronic hepatitis C patients, it inhibits GPT levels from increasing due to the discontinuation of interferon therapy, thus extending the effect of interferon (See M. Angelico et al., Amer. J. Gastroenterol. 90, 263-269 (1995)). In addition, ursodeoxycholic acid is a useful therapeutic agent that can significantly lowers liver enzyme levels after interferon therapy to reduce the recurrence rate of hepatitis (See C. Clerici et al., Minerva Med. 88, 219-225 (1997)).

Patent applications relating to the use of ursodeoxycholic acid as an agent for treating hepatitis C were filed in Korea, and International Application No. PCT/JP94/001178 discloses the use of ursodeoxycholic acid as an agent for inhibiting the proliferation of hepatitis C virus. However, it has been reported in a number of studies that ursodeoxycholic acid does not directly inhibit hepatitis C virus (See Ursodeoxycholic acid ‘mechanisms of action and clinical use in hepatobiliary disorders’. Lazaridis K N, Gores G J, Lindor K D. J. Hepatol., July 2001, 35(1):134-46).

Among prior patents, US Patent Publication No. 2005/0187204 discloses the use of a combination of an HMG-CoA reductase inhibitor and bile acid. However, the invention disclosed in said US Patent Publication merely aims at the treatment of diseases caused by hyperlipidemia and does not mention the treatment of hepatitis C virus infection which is the subject matter of the present invention.

### DISCLOSURE OF INVENTION

### Technical Problem

It is an object of the present invention to provide a combined preparation for treating viral hepatitis, which comprises an HMG-CoA reductase inhibitor useful as an agent that is administered in combination with an existing hepatitis C therapeutic agent in order to assist the hepatitis C therapeutic agent or hepatitis C therapeutic effects.

The present inventors have developed a functional combined preparation which comprises a combination of an HMG-CoA reductase inhibitor and bile acid, and thus has an improved antiviral activity against hepatitis C and can be administered for a long-term period without side effects such as anemia caused by prior ribavirin, thereby completing the present invention.

### Technical Solution

The present invention provides a stable agent for treating hepatitis C virus infection, comprising an HMG-CoA reductase inhibitor and bile acid, and a preparation method thereof. More specifically, the present invention provides an agent for treating hepatitis C virus infection, comprising fluvastatin as an HMG-CoA reductase inhibitor and ursodeoxycholic acid as bile acid, and a preparation method thereof.

Specifically, an aspect of the present invention relates to:

(1) a pharmaceutical composition for treating hepatitis C virus infection, comprising an HMG-CoA reductase inhibitor and bile acid as active ingredients;

(2) the pharmaceutical composition of the above (1), wherein the HMG-CoA reductase inhibitor comprises one or more selected from the group consisting of fluvastatin, lovastatin, atorvastatin, simvastatin, livastatin, pitavastatin, rosuvastatin, and salts thereof;

(3) the pharmaceutical composition of the above (2), wherein the HMG-CoA reductase inhibitor is fluvastatin or a salt thereof;

(4) the pharmaceutical composition of the above (3), wherein the salt of fluvastatin is a sodium salt;

(5) the pharmaceutical composition of the above (1), wherein the bile acid comprises one or more selected from the group consisting of ursodeoxycholic acid, chenodeoxycholic acid, deoxycholic acid, cholic acid, and salts thereof;

(6) the pharmaceutical composition of the above (5), wherein the bile acid is ursodeoxycholic acid or a salt thereof;

(7) the pharmaceutical composition of the above (1), wherein the amount of the HMG-CoA reductase inhibitor is in the range of 0.05-200 mg, and the amount of the bile acid is in the range of 10-1,500 mg;

(8) the pharmaceutical composition of the above (7), wherein the amount of the HMG-CoA reductase inhibitor is in the range of 0.1-100 mg, and the amount of bile acid is in the range of 25-1,000 mg;

(9) the pharmaceutical composition of the above (1), which further comprises interferon;

(10) the pharmaceutical composition of the above (9), wherein the amount of interferon is in the range of 1000-1,000,000,000 units;

(11) the pharmaceutical composition of the above (10), wherein the amount of the interferon is in the range of 10,000-100,000,000 units;

(12) the pharmaceutical composition of the above (1), which is in the form of a two-phase matrix, a multilayered tablet or a press-coated tablet;

(13) the pharmaceutical composition of the above (1), which is in the form of a capsule comprising two-phase granules consisting of delayed-release granules and immediate-release granules;

(14) the pharmaceutical composition of the above (1), which is in the form of a multilayered tablet;

(15) a combination preparation for treating hepatitis C virus infection comprising an HMG-CoA reductase inhibitor and bile acid as active ingredients;

(16) a method for preventing or treating hepatitis C virus infection, which comprises administering a pharmaceutical composition comprising an HMG-CoA reductase inhibitor and bile acid as active ingredients;

(17) the method of the above (16), wherein the pharmaceutical composition further comprises interferon;

(18) a method for preventing or treating hepatitis C virus infection, which comprises administering, together with interferon, a pharmaceutical composition comprising an HMG-CoA reductase inhibitor and bile acid as active ingredients;

(19) use of a pharmaceutical composition comprising an HMG-CoA reductase inhibitor and bile acid as active ingredients for preventing or treating hepatitis C virus infection; and

(20) the use of the above (19), wherein the pharmaceutical composition further comprises interferon as an active ingredient.

### Advantageous Effects

The inventive pharmaceutical composition comprising an HMG-CoA reductase inhibitor and bile acid may substitute for ribavirin which has been used in combination with interferon in the prior art. Also, the inventive pharmaceutical composition may be used as an additional component in a combination therapy of interferon and ribavirin which has been used for the treatment of hepatitis C virus infection in the prior art.

The inventive pharmaceutical composition has no anemia-related side effects caused by ribavirin, and thus may be administered for a long-term period. In addition, it can ameliorate the conditions of patients with chronic liver diseases including hepatitis C virus infection.

### DESCRIPTION OF DRAWINGS

FIG. 1 shows the construction of a 96-well plate used in Test Example 1.

FIG. 2 is a graphic diagram showing the inhibitory effects of interferon α-2b, fluvastatin, interferon+fluvastatin and interferon+fluvastatin+ursodeoxycholic acid against hepatitis C virus, evaluated in Test Example 1.

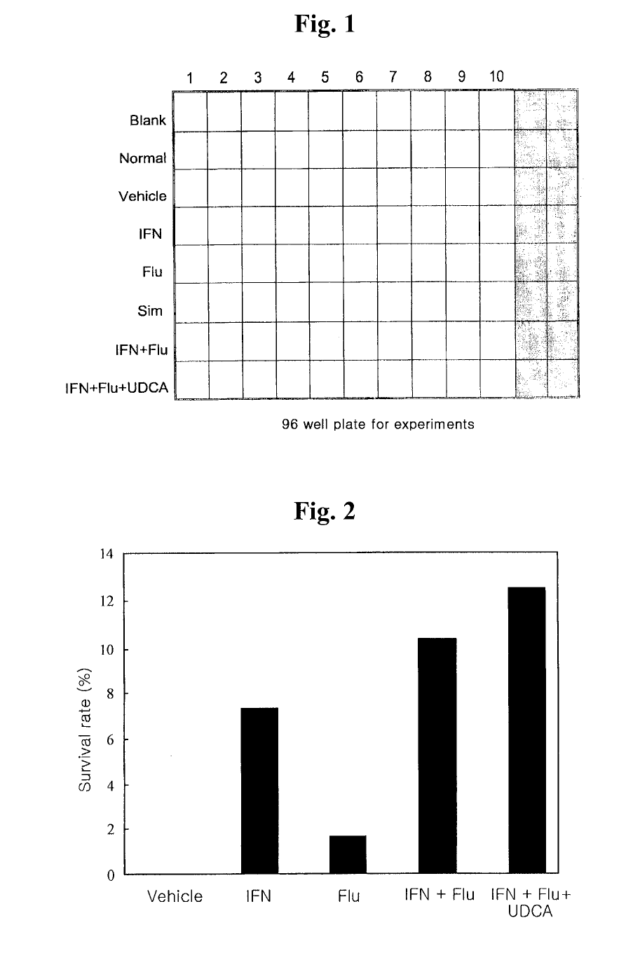

### BEST MODE

The present invention relates to a pharmaceutical composition comprising, as active ingredients, an HMG-CoA reductase inhibitor which has hepatitis C inhibitory effects, and bile acid which has a significant synergistic action for the hepatitis C inhibitory effect along with liver function-improving effects, and a preparation method thereof. More specifically, the present invention relates to a pharmaceutical composition comprising a combination of fluvastatin, as an HMG-CoA reductase inhibitor, and ursodeoxycholic acid having a significant synergistic action for hepatitis C inhibitory effects along with liver function-improving effects, as bile acid, and a preparation method thereof.

The combined preparation of fluvastatin and ursodeoxycholic acid according to the present invention shows an inhibitory effect against hepatitis C virus, and thus may substitute for ribavirin, which has been used as a hepatitis C therapeutic agent for oral administration and has side effects such as anemia. Also, it may be used as a new therapeutic agent for patients in which the prior combined therapy of interferon and ribavirin does not have sufficient effects. In Test Example 1 below, the inhibitory effects of fluvastatin, ursodeoxycholic acid and interferon against hepatitis C virus were evaluated.

As can be seen in Test Example 1, the pharmaceutical composition of the present invention had an inhibitory effect against hepatitis C virus. Also, it was seen that the drugs of the pharmaceutical composition of the present invention had a high synergistic effect when they were administered in combination as compared to when they were administered alone.

In order to apply the composition of the present invention to hepatitis C patients in an easier manner, a combined preparation comprising fluvastatin and ursodeoxycholic acid may be used, and for more efficient application, a combined preparation comprising interferon, fluvastatin and ursodeoxycholic acid may be used. Also, a combined preparation containing either interferon and fluvastatin or interferon and ursodeoxycholic acid can be developed and used for the treatment of hepatitis C virus infection.

In the case of fluvastatin, it is excreted through the biliary pathway and likely to undergo significant first-pass metabolism in the liver. For this reason, the accumulation of fluvastatin in hepatitis C patients having reduced liver functions can occur. Thus, in order to administer fluvastatin to patients in a safer manner, it is required to control the release rate of fluvastatin in vivo. When the release rate of fluvastatin is controlled, the amount per hour of fluvastatin undergoing first-pass metabolism can be reduced, leading to a decrease in the total availability or peak blood concentration of the drug, but it is possible to administer fluvastatin in a safer manner, because a load on hepatic metabolism in patients can be reduced. In addition, the peak blood concentration fluvastatin in vivo increases by geometric progression with an increase in the dose thereof, and for this reason, a preparation having controlled release of fluvastatin is safer, even when the dose of fluvastatin is increased for the effective treatment of hepatitis C.

Meanwhile, ursodeoxycholic acid is absorbed in vivo by passive diffusion, but the absorption rate thereof is low and about 50% of the absorbed drug is excreted in the form of bile. However, the unabsorbed drug is subjected to the conjugation, deconjugation and resorption processes through enterohepatic circulation, and in the case of liver disease patients, the amount thereof excreted through bile decreases, and thus the amount thereof transmitted to the whole body increases compared to in the case in which normal hepatic metabolism occurs. Thus, in the case of ursodeoxycholic acid, it is advantageous in terms of medicinal efficacy to allow the drug to be released rapidly in vivo after administration so as to undergo continued enterohepatic circulation rather than additionally controlling the release of the drug within the preparation containing the drug.

Accordingly, the novel composition of the present invention comprises: a delayed-release section comprising an HMG-CoA reductase inhibitor or a pharmaceutically acceptable salt thereof and a pharmaceutically acceptable conventional excipient; and an immediate release section comprising bile acid and a pharmaceutically acceptable conventional excipient, wherein the two sections are physically separated or divided from each other, such that the two drugs may have different release rates. The inventive composition having such physical sections can be prepared in various formulations. For example, the inventive composition can be prepared in various formulations, including a two-phase matrix tablet having two divided granular phases within a single tablet, a multilayered tablet, a press-coated tablet, and a capsule formulation filled with two-phase granules consisting of delayed-release granules and immediate release granules, and the scope of the present invention is not limited thereto.

According to the present invention, the two-phase matrix tablet is characterized in that it comprises the following elements:

a) a delayed-release layer comprising particles or granules obtained by subjecting HMG-CoA reductase inhibitor and a release-controlling substance selected from the group consisting of an enteric polymer, a water-insoluble polymer, a hydrophobic compound and a hydrophilic polymer together with a pharmaceutically acceptable conventional excipient to a mixing, granulation or coating process; and

b) an immediate release layer obtained by subjecting bile acid and a pharmaceutically acceptable excipient to conventional processes for producing oral solid preparations, for example, mixing, kneading, drying and granulation.

The above-described preparation is a two-phase, controlled-release preparation in which the delayed-release layer shown in a) is surrounded by the immediate release layer shown in b).

The HMG-CoA reductase inhibitor may be used in the pharmaceutically acceptable form. Preferably, fluvastatin and a sodium salt of fluvastatin may be used. However, the scope of the present invention is not limited to fluvastatin.

The bile acid may be used in the pharmaceutically acceptable form. Most preferably, ursodeoxycholic acid may be used. However, the scope of the present invention is not limited to ursodeoxycholic acid.

In the process of preparing the delayed-release layer or immediate release layer of the formulation, interferon may be added and fluvastatin or ursodeoxycholic acid may be replaced with interferon. Interferon may be used in the pharmaceutically acceptable form. Preferably, interferon α-2b may be used.

The amount of the HMG-CoA reductase inhibitor per tablet is in the range of 0.05 mg to 200 mg, and the amount of the bile acid per tablet is in the range of 10 mg to 1,500 mg. Preferably, the amount of the HMG-CoA reductase inhibitor per tablet is in the range of 0.1 mg-100 mg, and the amount of bile acid per tablet is in the range of 25 mg to 1,000 mg. The amount of interferon per tablet is in the range of 1000 units to 1,000,000,000 units in terms of efficacy, and preferably in the range of 10,000 units to 100,000,000 units.

As the enteric polymer, one or more selected from the group consisting of polyvinyl acetate phthalate, methacrylic acid copolymers, hydroxypropylmethylcellulose phthalate, shellac, cellulose acetate phthalate and cellulose propionate phthalate may be used. Preferably, hydroxypropylmethylcellulose phthalate is used.

The water-insoluble polymer may be selected from the group consisting of polyvinyl acetate, polymethacrylate copolymers, such as poly(ethylacrylate, methylmethacrylate) copolymers and poly(ethylacrylate, methyl methacrylate, trimethylaminoethylmethacrylate) copolymers, ethyl cellulose, cellulose acetate and the like, which are pharmaceutically acceptable.

The hydrophobic compound may be selected from the group consisting of: fatty acids or fatty acid esters, including glyceryl palmitostearate, glyceryl stearate, glyceryl behenate, cetyl palmitate, glyceryl monooleate, stearic acid and the like; fatty acid alcohols, including cetostearyl alcohol, cetyl alcohol, stearyl alcohol and the like; waxes, including Carnauba wax, beewax, microcrystalline wax and the like; and inorganic materials, including talc, precipitated calcium carbonate, calcium hydrogen phosphate, zinc oxide, titanium oxide, kaolin, bentonite, montmorillonite, veegum and the like.

The hydrophilic polymer may be selected from the group consisting of: saccharides, including dextrin, polydextrin, dextran, pectin and pectin derivatives, alginate, polygalacturonic acid, xylan, arabinoxylan, arabinogalactan, starch, hydroxypropyl starch, amylose, amylopectin and the like; cellulose derivatives, including hydroxypropyl cellulose, hydroxymethyl cellulose, hydroxypropylmethyl cellulose, hydroxyethyl cellulose, methyl cellulose, carboxymethyl cellulose sodium, hydroxypropyl methylcellulose acetate succinate, hydroxyethylmethylcellulose and the like; gums, including guar gum, locust bean gum, tragacanth, carrageenan, gum acacia, gum arabic, gellan gum, xanthan gum and the like; proteins, including gelatin, casein, zein and the like; polyvinyl derivatives, including polyvinyl alcohol, polyvinyl pyrrolidone, polyvinylacetal diethylaminoacetate and the like; polymethacrylatc copolymers, including poly(butyl methacrylate, (2-dimethylaminoethyl)methacrylatc, methylmethacrylate) copolymers, poly(methacrylic acid, methylmethacrylate) copolymers, poly(methacrylic acid, ethylacrylate) copolymers and the like; polyethylene derivatives, including polyethylene glycol, polyethylene oxide and the like; and carboxyvinyl polymers such as carbomer.

Hereinafter, each step of a method for preparing the inventive pharmaceutical comprising an HMG-CoA reductase inhibitor and bile acid will be described in detail.

Step 1 is a step of obtaining a delayed-release composition comprising particles or granules obtained by subjecting an HMG-CoA reductase inhibitor or a pharmaceutically acceptable salt thereof as an active ingredient and a release-controlling substance selected from the group consisting of an enteric polymer, a water-insoluble polymer, a hydrophobic compound, a hydrophilic polymer and the like together with a pharmaceutically acceptable conventional excipient to a mixing, kneading, drying, granulation or coating process.

Step 2 is a step of obtaining an immediate release composition comprising particles or granules obtained by subjecting bile acid as an active ingredient together with a pharmaceutically acceptable excipient to conventional processes for producing oral solid preparations, for example, mixing, kneading, drying and granulation. If the fluidity of the bile acid mixture is good such that it can be directly compressed into tablets, the composition can be obtained through a mixing process, and if the fluidity is poor, the composition can be obtained in the form of granules by compression, granulation and sieving.

Step 3 is a step of obtaining a preparation for oral administration by post mixing the particles or granules obtained in each of steps 1 and 2 with a pharmaceutically acceptable excipient and either compressing the mixture into a tablet or filling the mixture in a capsule for oral administration.

According to the above-described method, the two-phase matrix tablet having controlled release of the HMG-CoA reductase inhibitor and bile acid is formed. However, a controlled-release preparation which can be provided according to the present invention is not limited to the two-phase matrix tablet which exists as a single tablet in which the delayed-release layer of the HMG-CoA reductase inhibitor locates in the immediate release layer of bile acid.

A tablet for oral administration which shows immediate release and delayed-release according to each layer therein may be obtained by mixing the granules obtained in steps 1 and 2 with pharmaceutical excipients and compressing the mixture using a multiple tableting machine into a double-layer or triple-layer tablet, the layers of which are parallel to each other.

Also, a tablet for oral administration which has an immediate release layer as an outer layer and a core layer as a delayed-release layer can be obtained by mixing the granules obtained in step 1 with a pharmaceutical excipient, compressing the mixture into a core tablet, mixing the granules obtained in step 2 with a pharmaceutical excipient, and then compressing the mixture onto the core tablet to form an outer layer.

Furthermore, a capsule preparation for oral administration can be obtained by mixing the granules obtained in steps 1 and 2 with pharmaceutical excipients, if necessary, and filling the granules in a capsule.

In addition to the above-described active ingredients and release-controlling agents, pharmaceutically acceptable dilutes, such as starch, microcrystalline cellulose, lactose, glucose, mannitol, alginate, alkaline earth metal salts, polyethylene glycol, dicalcium phosphate and the like, may be used in the tablet layer, as long as they do not impair the effects of the present invention. As binders, starch, microcrystalline cellulose, highly dispersible silica, mannitol, lactose, polyethylene glycol, polyvinyl pyrrolidone, hydroxypropylcellulose, natural gum, synthetic gum, Copovidone, gelatin and the like may be used in the inventive composition. As disintegrants, starches or modified starch such as sodium starch glycolate, corn starch, potato starch, pregelatinized starch, clays such as bentonite, montmorillonite, veegum and the like, cellulose such as microcrystalline cellulose, hydroxypropylcellulose, carboxymethylcellulose and the like, alginics such as sodium alginate, alginic acid and the like, cross-linked cellulose such as croscarmellose sodium and the like, crosslinked polymers such as crospovidone and the like, and effervescent additives such as sodium bicarbonate, citric acid and the like, may be used in the inventive composition. As lubricants, talc, alkali earth metal stearate, lauryl sulfate, hydrogenated vegetable oil, sodium benzoate, sodium stearyl fumarate, glyceryl monostearate, polyethyleneglycol 400 and the like may be used in the composition of the present invention. In addition, pharmaceutically acceptable additives selected from among colorants, fragrances and the like may be used in the present invention.

As such additives, microcrystalline cellulose, sodium starch glycolate, sodium lauryl sulfate, magnesium stearate and the like were used in Examples of the present invention, but the scope of the present invention is not limited to the use of these additives, and these additives may be contained in suitable amounts selected by a skilled person in the art.

Also, if necessary, a film coating layer may be formed on the outer surface of the tablet.

The inventive composition comprising an HMG-CoA reductase inhibitor and bile acid may also be used in the form of an uncoated tablet. However, when a coating layer is formed on the surface of the tablet layer containing the active ingredients, there is an advantage in that the stability of the active ingredients can be further ensured.

A method for forming the coating layer may be suitably selected by a skilled person in the art. from among methods capable of forming a film coating layer on the surface of the tablet layer using the above-described active ingredients. Preferably, a fan coating method may be used.

The coating layer may be made of a film-forming agent, a film-forming aid or a mixture thereof. Specifically, the film-forming agent for the coating layer may be one or a mixture of two or more selected from cellulose derivatives, saccharide derivatives, polyvinyl derivatives, waxes, fats, gelatin and the like, and the film-forming aid may be one or a mixture of two or more selected from the group consisting of polyethylene glycol, ethylcellulose, glycerides, titanium oxide and diethyl terephthalate.

When the inventive composition is formulated into a coated tablet, the coating layer is preferably contained in an amount of 0.5-15 wt % based on the total weight of the tablet.

### EXAMPLES

Hereinafter, the present invention will be described in detail with reference to examples, but the scope of the present invention is not limited to these examples.

### Example 1

### Preparation of Pharmaceutical Composition in Form of a Multilayered Tablet Comprising Fluvastatin and Ursodeoxycholic Acid

1) Preparation of Sustained-Release Granules of Fluvastatin

According to the components and contents shown in Table 1 below, fluvastatin sodium salt, microcrystalline cellulose and Povidone were sieved through sieve No. 20, and then mixed with each other in a high-speed mixer for 3 minutes. The mixture of the main components in the high-speed mixer was kneaded with purified water for 3 minutes, and then dried in a fluidized-bed dryer at 90° C., until the LOD thereof reached less than 2%. Then, the dried mixture was sieved using an oscillator equipped with sieve No. 20. Hydroxypropylcellulose sieved through sieve No. 35 was added to and mixed with the mixture. Finally, magnesium stearate sieved through sieve No. 35 was added to and mixed with the mixture.

2) Preparation of Ursodeoxycholic Acid Granules

According to the components and contents shown in Table 1 below, hydroxypropylcellulose was dissolved in purified water to prepare a binder solution. Meanwhile, ursodeoxycholic acid, microcrystalline cellulose and maize starch were sieved through sieve No. 20, and then mixed with each other in a high-speed mixer for 3 minutes. The mixture of the main components in the high-speed mixer was kneaded with the binder solution for 3 minutes, and then sieved using an oscillator equipped with sieve No. 20. The sieved mixture was dried in a fluidized-bed dryer at 90° C., until the LOD thereof reached not more than 2%. The dried mixture was sieved using an oscillator equipped with sieve No. 20. Sodium lauryl sulfate and sodium starch glycolate were sieved through sieve No. 35, and then mixed with the mixture. Finally, magnesium stearate sieved through sieve No. 35 was added to and mixed with the mixture.

3) Tableting and Coating

Tableting was carried out using a multilayer tableting machine (MRC-37T, Sejong Pharmatech Co., Ltd., Korea). The composition containing ursodeoxycholic acid was placed in a first powder feeder, and the sustained-release layer composition containing fluvastatin was placed in a second powder feeder. The compositions were compressed into a tablet in conditions in which the incorporation between layers could be minimized. A film coating layer was formed on the tablet using a Hi-coater (SFC-30N, Sejong Pharmatech Co., Ltd.), thus preparing a multilayered sustained-release tablet.

### Example 2

### Preparation of Pharmaceutical Composition in Form of a Multilayered Tablet Comprising Fluvastatin and Ursodeoxycholic Acid

1) Preparation of Sustained-Release Granules of Fluvastatin

According to the components and contents shown in Table 1 below, fluvastatin sodium salt, microcrystalline cellulose, Povidone and hydroxypropylmethylcellulose were sieved through sieve No. 20 and mixed with each other in a double-cone mixer for 60 minutes. The mixture was slugged by roller compacting at a pressure of 15-20 MPa. Then, the mixture was sieved using an oscillator equipped with sieve No. 14. Finally, magnesium stearate sieved through sieve No. 35 was added to and mixed with the mixture.

2) Preparation of Ursodeoxycholic Acid Granules

According to the components and contents shown in Table 1 below, hydroxypropylcellulose was dissolved in purified water to prepare a binder solution. Meanwhile, ursodeoxycholic acid, microcrystalline cellulose and maize starch were sieved through sieve No. 20, and then mixed with each other in a high-speed mixer for 3 minutes. The mixture of the main components in the high-speed mixer was kneaded with the binder solution for 3 minutes, and then sieved using an oscillator equipped with sieve No. 20. The sieved mixture was dried in a fluidized-bed dryer at 90° C., until the LOD thereof reached not more than 2%. The dried mixture was sieved using an oscillator equipped with sieve No. 20. Sodium lauryl sulfate and sodium starch glycolate were sieved through sieve No. 35, and then mixed with the mixture. Finally, magnesium stearate sieved through sieve No. 35 was added to and mixed with the mixture.

3) Tableting and Coating

Tableting was carried out using a multilayer tableting machine (MRC-37T, Sejong Pharmatech Co., Ltd., Korea). The composition containing ursodeoxycholic acid was placed in a first powder feeder, and the sustained-release layer composition containing fluvastatin was placed in a second powder feeder. The compositions were compressed into a tablet in conditions in which the incorporation between layers could be minimized. A film coating layer was formed on the tablet using a Hi-coater (SFC-30N, Sejong Pharmatech Co., Ltd.), thus preparing a multilayered sustained-release tablet.

### Example 3

### Preparation of Pharmaceutical Composition in Form of Capsule Comprising Fluvastatin and Ursodeoxycholic Acid

1) Preparation of Sustained-Release Granules of Fluvastatin

According to the components and contents shown in Table 1 below, fluvastatin sodium salt, microcrystalline cellulose, Povidone, polyethylene oxide and xanthan gum were sieved through sieve No. 20, and then mixed with each other in a high-speed mixer for 3 minutes. The mixture of the main components in the high-speed mixer was kneaded with purified water for 3 minutes, and then dried in a fluidized-bed dryer at 90° C., until the LOD thereof reached less than 2%. Then, the dried mixture was sieved using an oscillator equipped with sieve No. 20.

2) Preparation of Ursodeoxycholic Acid Granules

According to the components and contents shown in Table 1 below, hydroxypropylcellulose was dissolved in purified water to prepare a binder solution. Meanwhile, ursodeoxycholic acid, microcrystalline cellulose, maize starch, sodium lauryl sulfate and sodium starch glycolate were sieved through sieve No. 20, and then mixed with each other in a high-speed mixer for 3 minutes. The mixture of the main components in the high-speed mixer was kneaded with the binder solution for 3 minutes, and then sieved using an oscillator equipped with sieve No. 20. The sieved mixture was dried in a fluidized-bed dryer at 90° C., until the LOD thereof reached not more than 2%. The dried mixture was sieved using an oscillator equipped with sieve No. 20.

3) Post-Mixing and Filling in Capsule

The granule compositions were mixed with each other in a double-cone mixer. According to the components and contents shown in Table 1 below, light anhydrous silicic acid sieved through sieve No. 35 and added to and mixed with the mixture. Then, magnesium stearate sieved through sieve No. 35 was added to and mixed with the mixture. The resulting mixture was placed in a powder feeder and filled in capsule No. 0 using a capsule filling machine.

### Example 4

### Preparation of Two-Phase Matrix Preparation Comprising Fluvastatin and Ursodeoxycholic Acid

1) Preparation of Sustained-Release Granules of Fluvastatin

According to the components and contents shown in Table 1 below, fluvastatin sodium salt, microcrystalline cellulose, Povidone and ethylcellulose were sieved through sieve No. 20, and then mixed with each other in a high-speed mixer for 3 minutes. The mixture of the main components in the high-speed mixer was kneaded with purified water for 3 minutes, and then dried in a fluidized-bed dryer at 90° C., until the LOD thereof reached less than 2%. Then, the dried mixture was sieved using an oscillator equipped with sieve No. 20.

2) Preparation of Ursodeoxycholic Acid Granules

According to the components and contents shown in Table 1 below, hydroxypropylcellulose was dissolved in purified water to prepare a binder solution. Meanwhile, ursodeoxycholic acid, microcrystalline cellulose and maize starch were sieved through sieve No. 20, and then mixed with each other in a high-speed mixer for 3 minutes. The mixture of the main components in the high-speed mixer was kneaded with the binder solution for 3 minutes, and then sieved using an oscillator equipped with sieve No. 20. The sieved mixture was dried in a fluidized-bed dryer at 90° C., until the LOD thereof reached not more than 2%. The dried mixture was sieved using an oscillator equipped with sieve No. 20. Sodium lauryl sulfate and sodium starch glycolate were sieved through sieve No. 35, and then mixed with the mixture.

3) Tableting and Coating

The granule compositions were mixed with each other in a double-cone mixer. According to the components and contents shown in Table 1 below, light anhydrous silicic acid was sieved through sieve No. 35, and then mixed with the mixture. Then, magnesium stearate was sieved through sieve No. 35 and mixed with the mixture. The mixture was compressed into a tablet using a rotary tableting machine (MRC-33, Sejong Pharmatech Co., Ltd.), and a film coating layer was formed on the tablet using a Hi-coater (SFC-30N, Sejong Pharmatech Co., Ltd.), thus preparing a sustained-release matrix tablet.

### Example 5

### Preparation of Pharmaceutical Composition in Form of Press-Coated Tablet Comprising Fluvastatin and Ursodeoxycholic Acid

1) Preparation of Sustained-Release Core Tablet of Fluvastatin

According to the components and contents shown in Table 1 below, fluvastatin sodium salt, microcrystalline cellulose and Povidone were sieved through sieve No. 20, and then mixed with each other in a high-speed mixer for 3 minutes. The mixture of the main components in the high-speed mixer was kneaded with purified water for 3 minutes, and then dried in a fluidized-bed dryer at 90° C., until the LOD thereof reached less than 2%. Then, the dried mixture was sieved using an oscillator equipped with sieve No. 20. Then, hydroxypropylmethylcellulose sieved through sieve No. 35 was added to and mixed with the mixture. Then, magnesium stearate sieved through sieve No. 35 was added to and mixed with the mixture. Then, the mixture was compressed into a core tablet using a rotary tableting machine (MRC-33, Sejong Pharmatech Co., Ltd.).

2) Preparation of Ursodeoxycholic Acid Granules

According to the components and contents shown in Table 1 below, hydroxypropylcellulose was dissolved in purified water to prepare a binder solution. Meanwhile, ursodeoxycholic acid, microcrystalline cellulose and maize starch were sieved through sieve No. 20, and then mixed with each other in a high-speed mixer for 3 minutes. The mixture of the main components in the high-speed mixer was kneaded with the binder solution for 3 minutes, and then sieved using an oscillator equipped with sieve No. 20. The sieved mixture was dried in a fluidized-bed dryer at 90° C., until the LOD thereof reached not more than 2%. The dried mixture was sieved using an oscillator equipped with sieve No. 20. Sodium lauryl sulfate and sodium starch glycolate were sieved through sieve No. 35, and then mixed with the mixture. Then, magnesium stearate sieved through sieve No. 35 was added to and mixed with the mixture.

3) Tableting and Coating

The compositions were compressed using a press-coated tablet producing machine (RUD-1: Kilian) into a tablet comprising, as an inner core, the sustained-release tablet of fluvastatin, and as an outer layer, the composition comprising ursodeoxycholic acid. A film coating layer was formed on the tablet using a Hi-coater (SFC-30N, Sejong Pharmatech Co., Ltd.), thus preparing a sustained-release tablet in form of a press-coated tablet.

### Example 6

### Preparation of Pharmaceutical Composition in Form of a Multilayered Tablet Comprising Fluvastatin and Chenodeoxycholic Acid

According to the components and contents shown in Table 1 below, a pharmaceutical composition was prepared in the same manner as in Example 1, except that chenodeoxycholic acid was used instead of ursodeoxycholic acid.

### Example 7

### Preparation of Pharmaceutical Composition in Form of Multilayered Tablet Comprising Fluvastatin Deoxycholic Acid

According to the components and contents shown in Table 1 below, a pharmaceutical composition was prepared in the same manner as in Example 1, except that deoxycholic acid was used instead of ursodeoxycholic acid.

### Example 8

### Preparation of Pharmaceutical Composition in Form of Atorvastatin and Ursodeoxycholic Acid

According to the components and contents shown in Table 1 below, a pharmaceutical composition was prepared in the same manner as in Example 1, except that atorvastatin calcium salt was used instead of fluvastatin sodium salt.

### Example 9

### Preparation of Pharmaceutical Composition in Form of Multilayered Tablet Comprising Lovastatin and Ursodeoxycholic Acid

According to the components and contents shown in Table 1 below, a pharmaceutical composition was prepared in the same manner as in Example 1, except that lovastatin was used instead of fluvastatin sodium salt.

### Example 10

### Preparation of Pharmaceutical Composition in Form of Multilayered Tablet Comprising Pitavastatin and Ursodeoxycholic Acid

According to the components and contents shown in Table 1 below, a pharmaceutical composition was prepared in the same manner as in Example 1, except that pitavastatin calcium salt was used instead of fluvastatin sodium salt.

### Example 11

### Preparation of Pharmaceutical Composition in Form of Multilayered Tablet Comprising Rosuvastatin and Ursodeoxycholic Acid

According to the components and contents shown in Table 1 below, a pharmaceutical composition was prepared in the same manner as in Example 1, except that rosuvastatin calcium salt was used instead of fluvastatin sodium salt.

### Example 12

### Preparation of Pharmaceutical Composition in Form of Multilayered Tablet Comprising Simvastatin and Ursodeoxycholic Acid

According to the components and contents shown in Table 1 below, a pharmaceutical composition was prepared in the same manner as in Example 1, except that simvastatin was used instead of fluvastatin sodium salt.

### Comparative Example 1

### Preparation of Fluvastatin Tablet

According to the components and contents shown in Table 1 below, fluvastatin granules were prepared in the same manner as the method of Example 1 for preparing the sustained-release granules of fluvastatin. Then, the granules were compressed into a tablet using a rotary tableting machine (MRC-33, Sejong Pharmatech Co., Ltd.), and a film coating layer was formed on the tablet using a Hi-coater (SFC-30N, Sejong Pharmatech Co., Ltd.), thus preparing a sustained-release matrix tablet.

### Comparative Example 2

### Preparation of Ursodeoxycholic Acid Tablet

According to the components and contents shown in Table 1 below, ursodeoxycholic acid granules were prepared in the same manner as the method of Example 1 for preparing ursodeoxycholic acid granules. Then, the granules were compressed into a tablet using a rotary tableting machine (MRC-33, Sejong Pharmatech Co., Ltd.), and a film coating layer was formed on the tablet using a Hi-coater (SFC-30N, Sejong Pharmatech Co., Ltd.), thus preparing a sustained-release matrix tablet.

TABLE 1

| | | Combination (mg/tablet) | | | | | | | | | | | | | |
|---|---|---|---|---|---|---|---|---|---|---|---|---|---|---|---|
| | | Ex. | Ex. | Ex. | Ex. | Ex. | Ex. | Ex. | Ex. | Ex. | Ex. | Ex. | Ex. | Comp. | Comp. |
| Components | | 1 | 2 | 3 | 4 | 5 | 6 | 7 | 8 | 9 | 10 | 11 | 12 | Ex. 1 | Ex. 2 |
| Sustained | Fluvastatin | 84.24 | 84.24 | 84.24 | 84.24 | 84.24 | 84.24 | 84.24 | | | | | | 84.24 | |
| release | sodium | | | | | | | | | | | | | | |
| layer | Atorvastatin | | | | | | | | 86.76 | | | | | | |
| | calcium | | | | | | | | | | | | | | |
| | Lovastatin | | | | | | | | | 80.0 | | | | | |
| | Pitavastatin | | | | | | | | | | 4.0 | | | | |
| | calcium | | | | | | | | | | | | | | |
| | Rosuvastatin | | | | | | | | | | | 20.8 | | | |
| | Simvastatin | | | | | | | | | | | | 80.0 | | |
| | Microcrys- | 135.76 | 135.76 | 75.76 | 86.76 | 135.76 | 135.76 | 135.76 | 133.24 | 140.0 | 216.0 | 199.2 | 140.0 | 135.76 | |
| | talline | | | | | | | | | | | | | | |
| | calcium | | | | | | | | | | | | | | |
| | Povidone | 5.5 | 5.5 | 5.5 | 5.5 | 5.5 | 5.5 | 5.5 | 5.5 | 5.5 | 5.5 | 5.5 | 5.5 | 5.5 | |
| | Hydroxy- | 37.0 | 37.0 | | | 37.0 | 37.0 | 37.0 | 37.0 | 37.0 | 37.0 | 37.0 | 37.0 | 37.0 | |
| | methyl | | | | | | | | | | | | | | |
| | calcium | | | | | | | | | | | | | | |
| | Polyethylene | | | 25.0 | | | | | | | | | | | |
| | oxide | | | | | | | | | | | | | | |
| | Xanthangum | | | 37.0 | | | | | | | | | | | |
| | Ethyl | | | | 87.0 | | | | | | | | | | |
| | cellulose | | | | | | | | | | | | | | |
| | Magnesium | 2.5 | 2.5 | | | 2.5 | 2.5 | 2.5 | 2.5 | 2.5 | 2.5 | 2.5 | 2.5 | 2.5 | |
| | stearate | | | | | | | | | | | | | | |
| Fast | Ursodeoxy- | 300.0 | 300.0 | 300.0 | 300.0 | 300.0 | | | 300.0 | 300.0 | 300.0 | 300.0 | 300.0 | | 300.0 |
| release | cholic acid | | | | | | | | | | | | | | |
| layer | Chenodeoxy- | | | | | | 300.0 | | | | | | | | |
| | cholic acid | | | | | | | | | | | | | | |
| | Deoxycholic | | | | | | | 300.0 | | | | | | | |
| | acid | | | | | | | | | | | | | | |
| | Hydroxy- | 10.0 | 10.0 | 10.0 | 10.0 | 12.0 | 10.0 | 10.0 | 10.0 | 10.0 | 10.0 | 10.0 | 10.0 | | 10.0 |
| | propyl | | | | | | | | | | | | | | |
| | cellulose | | | | | | | | | | | | | | |
| | Microcrys- | 73.0 | 73.0 | 54.0 | 73.0 | 136.0 | 73.0 | 73.0 | 73.0 | 73.0 | 73.0 | 73.0 | 73.0 | | 73.0 |
| | talline | | | | | | | | | | | | | | |
| | cellulose | | | | | | | | | | | | | | |
| | Corn starch | 30.0 | 30.0 | 15.0 | 40.0 | 60.0 | 30.0 | 30.0 | 30.0 | 30.0 | 30.0 | 30.0 | 30.0 | | 30.0 |
| | Sodium | 2.0 | 2.0 | 2.0 | 2.0 | 2.0 | 2.0 | 2.0 | 2.0 | 2.0 | 2.0 | 2.0 | 2.0 | | 2.0 |
| | lauryl | | | | | | | | | | | | | | |
| | sulfate | | | | | | | | | | | | | | |
| | Sodium | 20.0 | 20.0 | 20.0 | 10.0 | 20.0 | 20.0 | 20.0 | 20.0 | 20.0 | 20.0 | 20.0 | 20.0 | | 20.0 |
| | starch | | | | | | | | | | | | | | |
| | glycolate | | | | | | | | | | | | | | |
| | Magnesium | 5.0 | 5.0 | | | 5.0 | 5.0 | 5.0 | 5.0 | 5.0 | 5.0 | 5.0 | 5.0 | | 5.0 |
| | stearate | | | | | | | | | | | | | | |
| Post | light | | | 1.5 | 1.5 | | | | | | | | | | |
| mixing | anhydrous | | | | | | | | | | | | | | |
| | silicic acid | | | | | | | | | | | | | | |
| | Magnesium | | | 5.0 | 5.0 | | | | | | | | | | |
| | stearate | | | | | | | | | | | | | | |
| | Total weight | 705.0 | 705.0 | 635.0 | 705.0 | 800.0 | 705.0 | 705.0 | 705.0 | 705.0 | 705.0 | 705.0 | 705.0 | 265.0 | 440.0 |
| | (mg/tablet) | | | | | | | | | | | | | | |

According to Example 3, a capsule formulation containing interferon in place of ursodeoxycholic acid can be prepared. Alternatively, granules containing ursodeoxycholic acid can be prepared using interferon as an additional component.

### Test Example 1

### Comparison of Inhibitory Effects on EMCV Between Drugs

1) Preparation of Test Solutions

Each of interferon α-2b, fluvastatin, ursodeoxycholic acid, interferon+fluvastatin and interferon+fluvastatin+ursodeoxycholic acid was diluted to the final concentration shown in Table 2 below.

TABLE 2

| Test groups | amount | N |
|---|---|---|
| Blank | — | 10 |
| Normal | — | |
| Vehicle | — | |
| IFN | 20 IU/ml | |
| Flu | 5 μM | |
| IFN + Flu | 20 IU/ml/5 μM | |
| IFN + Flu + UDCA | 20 IU/ml/5 μM/61.9 μM | |

Blank: Test medium (w/o cells)
Normal: Cell group uninfected by hepatitis C virus in test medium
Vehicle: Cell group infected by hepatitis C virus in test medium
IFN: Interferon α-2b
Flu: Fluvastatin
UDCA: Ursodeoxycholic acid

2) Cell Dispensing

A) 50 μL of each test solution was added to each well of a 96-well plate, and 50 μl of an experimental medium was added to each of wells for the blank, normal and vehicle groups. Then, the 96-well plate was incubated in a CO₂ incubator for 30 minutes.

B) A549 cells having a doubling time of 22 hours were suspended in an experimental medium at a concentration of 4×10⁵ cells/ml to a final volume of 20 ml.

C) 50 μL of the cell suspension was added to each of the wells (except for blank wells) of the plate of “A”, and 50 μL of an experimental medium was added to each of the blank wells. Then, the plate was incubated in CO₂ incubator for 24 hours.

3) Virus Infection

A) an EMC virus stock was diluted to a concentration of 5×10³ cells/ml (reconstitution).

B) 50 μl it of the dilution was added to each of the wells of all the test groups except for the blank and normal groups, and 50 μl, of an experimental medium was added to each of the blank and normal wells. Then, the plate was incubated in a CO₂ incubator for 24 hours.

4) MTT Assay

A) 15 μL of the MTT-labeling reagent was added to each well of the plate.

B) The plate was incubated in a CO₂ incubator for 4 hours.

C) 150 μL of a solubilizing solution was added to each well of the plate.

D) The plate was incubated in a CO₂ incubator for virus at 37° C. overnight (12-18 hours).

E) The production of purple formazan was measured and the absorbance was measured at a wavelength between 550 nm and 600 nm using a microplate reader.

Inhibitory effects on hepatitis C virus measured in Test Example 1 are shown in Table 3.

TABLE 3
Survival rate (antivirus activity)

| Test groups | Average | STDEV | Survival rate |
|---|---|---|---|
| Vehicle (including virus) | 0.8481 | 0.0401 | 0.0 |
| IFN 20 IU/ml | 0.9166 | 0.0522 | 7.4 |
| Fluvastatin 5 μM | 0.8638 | 0.0449 | 1.7 |
| IFN 20 IU/ml + Fluvastatin 5 μM | 0.9454 | 0.0224 | 10.5 |
| IFN 20 IU/ml + Fluvastatin 5 μM + | 0.9646 | 0.0352 | 12.5 |
| UDCA 61.9 μM | | | |

As can be seen in Table 3, an antiviral effect could be observed in all the test groups, even though the concentration of each of the test solutions used in Test Example 1 was 1/10 or 1/100 of those of the solutions used in the prior art. It could be seen that the test groups treated with each of interferon α-2b and fluvastatin showed a hepatitis C virus inhibitory effect which was about 1.4-fold higher than that of the test group treated only with interferon. The test group treated with only ursodeoxycholic acid showed no direct inhibition of hepatitis C virus as in the cases reported mainly after the year 2000, but the combined use of fluvastatin and ursodeoxycholic acid together with interferon showed a hepatitis C inhibitory effect which was about 1.7-fold higher than that in the test group treated only with interferon, and this combined use showed an unexpected additional inhibitory effect compared to the test group treated with fluvastatin in combination with interferon. In addition, when ursodeoxycholic acid was used in combination, low toxicity was shown in a cytotoxicity measurement test. The above results revealed that the inventive composition comprising fluvastatin and ursodeoxycholic acid can become an excellent therapeutic agent against hepatitis C virus infection.

1. A method for the prevention or treatment of hepatitis C viral infection, which comprises the administration of a therapeutically effect amount of a pharmaceutical composition comprising an HMG-CoA reductase inhibitor and bile acid as the active ingredients.

2. The method of claim 1, wherein the HMG-CoA reductase inhibitor comprises one or more inhibitors selected from the group consisting of fluvastatin, lovastatin, atorvastatin, simvastatin, livastatin, pitavastatin, rosuvastatin, and salts thereof.

3. The method of claim 2, wherein the HMG-CoA reductase inhibitor is fluvastatin or a salt thereof.

4. The method of claim 3, wherein the salt of fluvastatin is a sodium salt.

5. The method of claim 1, wherein the bile acid comprises one or more selected from the group consisting of ursodeoxycholic acid, chenodeoxycholic acid, deoxycholic acid, cholic acid, and salts thereof.

6. The method of claim 5, wherein the bile acid is ursodeoxycholic acid or a salt thereof.

7. The method of claim 1, wherein the amount of the HMG-CoA reductase inhibitor is in the range of 0.05 to 200 mg, and the amount of the bile acid is in the range of 10 to 1,500 mg.

8. The method of claim 7, wherein the amount of the HMG-CoA reductase inhibitor is in the range of 0.1 to 100 mg, and the amount of the bile acid is in the range of 25 to 1,000 mg.

9. The method of claim 1, wherein the composition further comprises interferon.

10. The method of claim 9, wherein the amount of interferon is in the range of 1000 to 1,000,000,000 units.

11. The method of claim 10, wherein the dose of the interferon is in the range of 10,000 to 100,000,000 units.

12. The method of claim 1, wherein the composition is formulated as a two-phase matrix, a multi-layered tablet or a press-coated tablet.

13. The method of claim 1, wherein the composition is formulated as a capsule comprising two-phase granules consisting of delayed-release granules and immediate-release granules.

14. The method of claim 12, wherein the composition is formulated as a multi-layered tablet.

15. A method for the prevention or treatment of hepatitis C viral infection, which comprises the simultaneous or sequential administration of therapeutically effective amount of a composition comprising an HMG-CoA reductase inhibitor and a composition comprising bile acid as active ingredients.

16-17. (canceled)

18. A method for the prevention or treatment of hepatitis C viral infection, which comprises the administration, together with interferon, of a pharmaceutical composition comprising an HMG-CoA reductase inhibitor and bile acid as the active ingredients.

19-20. (canceled)
